(19) United States
(12) Patent Application Publication
(10) Pub. No.: US 2015/0192357 A1
(43) Pub. Date: Jul. 9, 2015

(54) CONTROL OF FREEZING AND THAWING OF DRUG SUBSTANCES USING HEAT FLOW CONTROL

(71) Applicant: Millrock Technology, Inc., Kingston, NY (US)

(72) Inventor: Taylor N. THOMPSON, Jr., Kingston, NY (US)

(21) Appl. No.: 14/584,438

(22) Filed: Dec. 29, 2014

Related U.S. Application Data
(60) Provisional application No. 61/925,348, filed on Jan. 9, 2014.

(51) Int. Cl. F25D 29/00; F25D 31/00; F25D 17/00; A61K 9/19; F25D 3/00

(52) U.S. Cl. CPC F25D 29/00; A61K 9/19; F25D 3/005; F25D 17/00; F25D 31/001; F25D 2400/30; F25D 2700/00; F25D 2700/16

(57) ABSTRACT

A method of monitoring and controlling the freezing and thawing of a drug substance partially or fully surrounded by a temperature control medium. The heat flow between the drug substance and the temperature control medium is measured and controlled to create a uniform ice crystal structure to minimize any degradation of the drug substance being frozen and thawed.

### CROSS-REFERENCE TO RELATED APPLICATIONS

This application claims the priority of Provisional Patent Application No. 61/925,348 filed on Jan. 9, 2014.

### BACKGROUND OF THE INVENTION

1. Field of the Invention

The present invention relates to control of the freezing and thawing of drug substances and, more particularly, to the use of heat flow control of such freezing and thawing.

2. Description of the Background Art

Freezing and thawing of bulk materials for long term storage and use is commonly used for drug substances (IE: biological, enzymes, peptides, DNA, RNA, liposomes, proteins and other pharmaceutical products). Freezing stabilizes the material and reduces degradation that can occur at room temperature. Frozen material eliminates the risk of biological growth and elimination of agitation and foaming during shipment. The rate of freezing has a direct impact on the quality of product post-thaw. Frozen materials have to be thawed systematically for use after freezing. The thawing process can often be as important as the freezing process. For optimum product stability, the goal of the freezing and thawing method is to produce consistent and repeatable results.

There are several methods of freezing and thawing used. One flexible method is placing the material in a bag which is placed between 2 fluid filled plates. Another is placing the material in a large stainless vessel with fluid filled fins, called cryo-vessels. Each method controls the freezing by measuring and controlling the temperature control medium, such as a fluid or plate or fin. Controlling the heat transfer medium may produce a consistent result, but does not ensure a uniform ice crystal structure throughout the material since the ice crystal growth rate changes as a function of energy, not temperature. The rate of freezing can result in protein concentration gradients and shifts in buffer osmolality. In addition, in larger batches cryo-concentration can be a problem.

The rate of freezing has a dramatic impact on the viability of the product. Very slow freezing can cause solutes to be concentrated, which may cause degradation. Very rapid freezing can also cause protein loss. Small ice crystals are formed which causes aggregation and precipitation of proteins. Protein degradation is directly correlated to the surface area of the ice crystals formed. For proteins, an ideal freezing rate is where the ice surface area per volume ice is as low as possible without causing the solutes to concentrate. Freezing rate variability needs to be controlled to minimize the variation in cryo-concentration of excipients and protein. The robustness of a freezing process should minimize the heterogeneity within the frozen state.

Thawing is a more dynamic process and the method of control needs to minimize the heat transfer dynamics and to control the temperature and hold time of the final liquid.

The typical control method is open loop and controls the plate or circulating fluid temperature. To make the process work many experiments have to be performed and it still does not guarantee a robust solution. To improve the freezing and thawing processes a method for controlling the heat flow is required.

Freezing Process

Freezing of solutions occurs in three major stages; (a) nucleation, (b) crystallization of the equilibrium freeze concentrate, and ( ) concentration of the maximal freeze concentrate. The first two stages are crystal growth events. Each of these stages has its own unique challenges since the heat transfer from shelf to product changes dramatically and is not truly controlled.

Nucleation is the initial process of formation of a crystal in a super-cooled solution in which a small number of molecules become arranged in a stable structure upon which additional molecules are more easily deposited.

Once nucleation occurs, a portion of the water in the pre-lyophilized solution crystalizes. The amount of water that initially crystalizes depends on the degree of super-cooling and is typically between 3% and 19%. The initial ice propagation is limited to the capacity of solution and apparatus to absorb the latent heat of fusion of ice generated by the crystallization of water. As the ice grows from the nucleation sites, heat is released raising the temperature of the contents to approximately −0.5°. and crystal growth significantly slows.

Post-nucleation, the remaining unfrozen water, up to 97%, begins to crystalize as heat is further removed. Ice crystalizes from the equilibrium freeze concentrate as the shelf temperature is reduced and further energy is removed. The rate of crystal growth during this freezing stage is not well controlled and the ice forms at different rates creating a heterogeneous ice structure. The rate of ice crystal growth varies due to changes in heat flow.

The maximal freeze concentrate is formed when all the freezable water is converted to ice. The point where the maximal freeze concentrate is formed can be identified by the end of latent heat production. The maximal freeze concentrate either goes through freeze separation if it is a eutectic material or solidification if it is an amorphous material. Once the temperature of an amorphous product has been reduced below its glass transition temperature and the heat flow between the shelf and vial has reduced to a steady state condition, the product can be considered frozen and stable. The end of freezing can be identified when the heat flow approaches zero, indicating there is no longer a phase change or product temperature change.

Freezing in Bulk

Efficient freezing of materials reduces the negative effects of the freezing process, which include cryo-concentration, heterogeneity of ice crystals and protein concentration gradients, and shifts in buffer osmolality.

In the pharmaceutical world, freezing may be performed in bags that are immersed in a temperature control medium such as a liquid or placed between a medium such as two fluid filled plates or placed in a medium such as a large vessel with internal fluid filled fins. The product temperature is reduced and frozen by controlling the temperature of the plates, fluid, or walls in a predetermined temperature profile or recipe. The result of simply placing material in a bag or large vessel and reducing the plate, fluid, or wall temperature is heterogeneous freezing in the batch and potential degradation of the material.

Adding to the complexity of the process, the same process control temperature (fluid, plate, or wall) does not translate to a uniform heat flow to the material. In plate and wall style systems there can be a major variation in surface temperature from the inlet to the outlet. The temperature differential across the plate and walls varies by equipment design, fluid flow, type of fluid inside the shelf and the load on the shelf. The heat flow varies depending on plate and wall finish, fluid flow, bag type, and other variables. During temperature transition on a fully loaded system, the temperature difference across the plate or wall can be significant, for example greater than 10°. In fluid immersion systems sufficient mixing is required, however, adding bags to the fluid disturbs the flow and results in reduced temperature uniformity throughout.

During a controlled rate plate cool-down, the heat flow between the plate and the bag or product changes significantly. This can be observed using a heat flux sensor placed between the bag or product and the cooling/heating surface. The magnitude of heat flow increases as the temperature is dropped and then decreases as the latent heat is expended. The result is non-uniform crystal growth rate.

Using heat flow control may also enable control of the nucleation process which will produce a consistent initial ice crystal structure.

To overcome heterogeneous crystal structures created an annealing step is often added. Annealing is a process where the frozen product temperature is raised to allow the crystals and the interstitial pores to increase in size. Although this does help improve the crystal structure, if the frozen material is not uniform prior to annealing only marginal improvement can be achieved. Annealing can also potentially lead to changes in the protein structure.

### BRIEF SUMMARY OF THE INVENTION

Controlling heat flow enables control of the ice crystal growth during the entire freezing and thawing process and provides the tool necessary to scale from a small lab sized unit to a production sized unit. Using a heat flux sensor and a closed loop control method enable full control of the freezing (ice crystal growth) and thawing process as described hereinafter.

The challenge to monitoring for a proper crystal structure post-nucleation exists due to minimal product temperature change as the water changes phase from liquid to solid. Measuring product temperature provides no significant information about crystal growth. Measurement of the heat leaving the bag/product (heat flux) provides the information needed to monitor and control the ice crystal growth rate. The present invention provides a method to measure the heat flow between the heat transfer surface (plate, wall, fin or fluid) and the bag/product. Measuring the heat flux provides a new level of information previously unavailable to the freeze-thaw community about both the freezing and thawing processes.

Measuring the heat flow eliminates the guesswork of the time required to reach thermal equilibrium and reduces processing time.

Controlling the heat flow between the heat transfer surface and the bag/product is necessary to create a uniform ice crystal structure that minimizes degradation of the product being frozen. Using the present method, the heat transfer surface temperature can be adjusted to produce a consistent level of heat flow to/from the product. Controlling the heat flow throughout the entire freezing process, including super-cooling soak, crystallization of the freeze concentrate and solidification of the maximal freeze concentrate, the ice crystal structure can be controlled and a uniform structure intra-bag or intra-vessel is achieved. Controlling the freezing process in this fashion eliminates the effects of heat flow variations and allows re-modeling of the ice crystals. The result is significantly better ice crystal structures and better product uniformity.

### BRIEF DESCRIPTION OF THE DRAWINGS

FIG. 1 is a side elevational view in section showing one embodiment wherein a bag with drug substances therein is positioned between fluid filled plates and with heat flux sensors positioned between the bag and the plates;

FIG. 2 is schematic plan view showing a single heat flux sensor on or in a fluid filled plate or fin;

FIG. 3 is a schematic plan view showing multiple heat flux sensors mounted on or in a fluid filled plate or fin;

FIG. 4 is a side elevational view in section showing another embodiment wherein a bag with drug substances therein is immersed in a fluid bath and one or more heat flux sensors are mounted on the bag; and

FIG. 5 is a plan view in section of a further embodiment wherein a bulk drug substance is positioned within a vessel or tank having fluid filled fins therein.

### DETAILED DESCRIPTION OF THE INVENTION

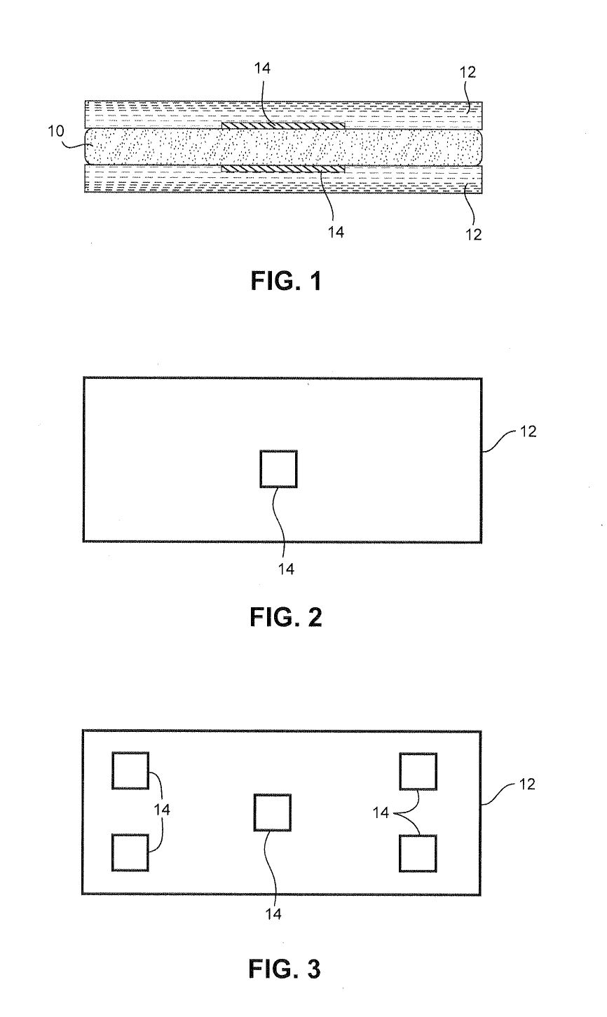

As shown in FIG. 1, one embodiment of apparatus for freezing and thawing of drug substances comprises a bag 10 containing the drug substances which is placed between two fluid filled plates 12 that serve as temperature control mediums for freezing the drug substance as is known in the prior art. In accordance with the method of the present invention, one or more heat flux sensors 14 of any suitable construction are positioned between the bag 10 and the plates 12 to measure the heat flow therebetween. The heat flux sensors 14 may be mounted on the surface of a plate 12 or may be embedded in the plate 12. Any suitable number of heat flux sensors 14 may be used and they may positioned on the plates 12 in any suitable manner. FIG. 2 illustrates a single heat flux sensor 14 mounted on a fluid filled plate 12 and FIG. 3 illustrates multiple heat flux sensors 14 mounted on a fluid filled plate 12.

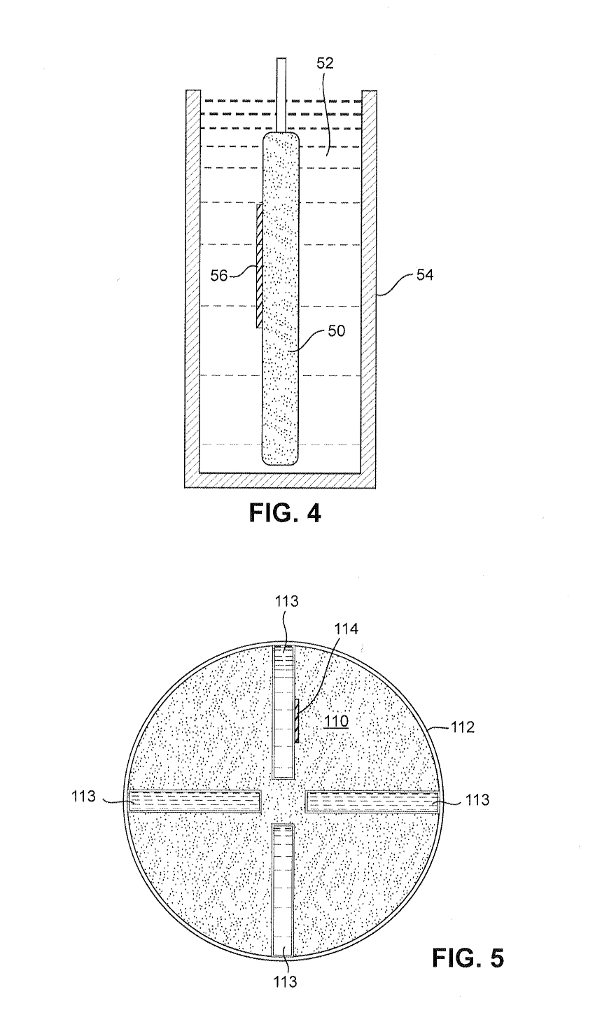

FIG. 4 illustrates another embodiment of apparatus for freezing and thawing of drug substances wherein a bag 50 containing the drug substance is immersed in a temperature controlled fluid 52 that serves as a temperature control medium for freezing the drug substance. The fluid 52 is contained within an enclosure 54 of any suitable type. One or more heat flux sensors 56 are mounted on or positioned in contact with the bag 50 to measure the heat flow between the drug substance and the fluid 52.

FIG. 5 illustrates another known apparatus for freezing and thawing drug substances in which a cryo-vessel or tank 112 formed of stainless steel or the like having one or more fluid filled fins 113, contains a bulk drug material 110 therein. One or more heat flux sensors 114 are positioned on the fins 113 to measure the heat flow between them and the drug material in the vessel 112. The fluid filled fins 113 serve as a temperature control medium for freezing the drug material. The vessel or tank 112 may also serve as a temperature control medium.

The method of the present invention is operable with the apparatus shown in FIGS. 1, 4 and 5, and with any other suitable apparatus for freezing and thawing of drug substances in which heat flow to and from the drug substances can be measured and controlled.

The ability to monitor and control the freezing process enables an operator to develop a fully controllable and repeatable ice and cake structure. Further testing and field results provide more information on the best heat flow control levels and profiles during freezing to produce the best pore structure for a specific product.

Another powerful feature of measuring the heat flow is that the end of freezing can be detected and any significant changes to the process, such as reduced fluid flow, can also be identified and corrected in-process.

The present method enables the K (bag/product conductivity) to be measured for a specific bag or drug product and fill. With the known sample conductivity (K), the measured heat flow information enables the product temperature to be determined during the freezing and thawing cycle, without the use of a thermocouple. The product temperature allows the plate/fin temperature to be controlled.

The present method provides a true real-time Process Analytical Technology without the use of thermocouples and other invasive techniques. For the first time the entire freezing process can be monitored and controlled. The measurement of heat flow in accordance with the present method enables control of both the freezing and drying processes which results in a significant reduction in processing time, a more uniform end product, and a method to qualify that the product has been processed properly.

Controlling the heat flow between the heat transfer surface such as a plate, fin or fluid and the bag/product is necessary to create a uniform ice crystal structure that minimizes degradation of the product being frozen. Using the present method, the heat transfer surface temperature can be adjusted to produce a consistent level of heat flow to/from the product. Controlling the heat flow throughout the entire freezing process, including super-cooling soak, crystallization of the freeze concentrate and solidification of the maximal freeze concentrate, the ice crystal structure can be controlled and a uniform structure intra-bag or intra-vessel is achieved. Controlling the freezing process in this fashion eliminates the effects of heat flow variations and allows re-modeling of the ice crystals. The result is significantly better ice crystal structures and better product uniformity.

Using heat flux measurement and control for freezing and thawing of bulk drug materials or other materials:

1—Controls ice crystal growth rates

2—Enables the determination of the end of freezing

3—Allows the product temperature to be calculated without the use of thermocouples

4—Enables the thawing process to be completed in a uniform fashion

5—Enables determination of the end of thawing

6—Is a true Process Analytical Technology for measurement on control of the process

7—Enables dynamic process control to compensate for variations in process such as changes in equipment or product heat transfer variables

8—Enables a lab sized unit to mimic the full-scale freezing rates for developing scale-up and transfer from the lab to production.

9—Enables smaller systems to mimic larger systems for scale-down

10—Enables the definition of a design space

11—Eliminates the guesswork of system failure. Heat transfer calculations based on the measurement can be used to verify the effect on the product.

12—Eliminates the change in process dynamics when operating equipment on different electrical frequencies, such as 50 Hz vs 60 Hz.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of monitoring and controlling a freezing and thawing of a drug substance partially or fully surrounded by a temperature control medium, comprising:
measuring the heat flow between the drug substance and the temperature control medium; and
controlling the heat flow to create a uniform ice crystal structure to minimize any degradation of the drug substance being frozen and thawed.

2. The method of claim 1 wherein the drug substance is positioned in a bag and the temperature control medium is a fluid filled plate positioned in contact with the bag.

3. The method of claim 2 wherein fluid filled plates are positioned on opposite sides of and in contact with the bag.

4. The method of claim 2 wherein a heat flux sensor is positioned between the bag and the fluid filled plate.

5. The method of claim 4 wherein a plurality of heat flux sensors are positioned between the bag and the fluid filled plate.

6. The method of claim 4 wherein the heart flux sensor is mounted on the fluid filled plate.

7. The method of claim 1 wherein the drug substance is contained within the temperature control medium in the form of a vessel with fluid filled fins.

8. The method of claim 7 wherein a heat flux sensor is mounted between the fluid filled fins and the drug substance.

9. The method of claim 8 wherein a heat flux sensor is mounted on one or more of the fluid filled fins.

10. The method of claim 1 wherein the temperature of the temperature control medium is adjusted in response to heat flow measurements to produce a consistent level of heat flow to or from the drug substance.

11. The method of claim 1 wherein the drug substance is positioned in a bag and the temperature control medium is a fluid in which the bag is immersed.

12. The method of claim 11 wherein a heat flux sensor is positioned between the bag and the temperature control fluid.

13. The method of claim 12 wherein the heat flux sensor is in contact with the bag.

14. The method of claim 13 wherein the heat flux sensor is mounted on the bag.

15. The method of claim 1 wherein a plurality of heat flux sensors are positioned between the bag and the temperature control fluid.

16. The method of claim 15 wherein the heat flux sensors are in contact with the bag.

17. The method of claim 16 wherein the heat flux sensors are mounted on the bag.
